SEQUENCE LISTING (2 sequences)

SEQ ID NO: 1 - DNA, 3577 bp, Canine
atggaggtacagttagggctagggagggtctacccccggccgccgtccaagacctatcgaggagctttccagaacctgttccagagtgtgcgcgaagtgatccagaacccgggccccaggcaccctgaggccgtgagcgcagcacctcccggtgcccatttgcagcagcagcagcagcagcagcagcagcaggagaccagtcctcggcagcagcagcagcaacagcagggtgacgatggctctccccaagcgcagagcagaggccccacaggctacctggctctggatgaggaacagcagccttcccaacagcggtcagcctccaagggccatccggagagtgcctgcgttccagagcctggagtgacttcggccaccggcaaggggctgcagcagcagcagccagcaccaccggacgagaatgactcagctgccccatccacattgtcactgctgggccccactttcccgggcttaagtagctgttccaccgatcttaaagacatcctgagcgaggctggaaccatgcaactccttcagcagcagcggcagcagcagcagcagcagcagcaacagcaacagcagcagcagcagcagcaacagcaggaggtagtatcagaaggtagcagcagcgggagagcaagggaggccgccggtgcttccacctcctccaaggacagttacctaggtggcagttcgaccatctcggacagcgccaaggagttgtgtaaggcagtgtcggtgtccatgggtttgggtgtggaggcattggaacatctgagccctggggaacagcttcggggggattgtatgtacgccccgctcctgggaggtccacccgccgtacgtccttgcgctccgctggccgaatgcaaaggttctctgctggatgacggcccgggcaagggcaccgaagaaactgctgaatattcccctttcaaggcaggttatgcgaaagggttggatggggacagcctgggctgttcgagcagcagtgaagcagggggctccggaacacttgagatgccatccaccctgtctctttacaagtctggagcactagatgaagcggcagcttatcaaagtcgagactactacaactttcctctctccctaggcgggccgcctccccatccaccacctccccatcctcacacccgcatcaagctggaaaaccctctggactatggcagcgcctgggcggctgcagcggcacaatgccgctacggggatctggcgagcctgcacggagcgggtgcagcaggacccagctcgggctcaccttcggccaccacctcctcttcctggcacactctcttcacagcagaagaaggccagctgtatgggccctgcggcgggagtggaggcggcagtgcaggcgacgggggatctgtagccccctatggctacactcggccacctcagggattggcgggtcaggaaggtgacttccctccacctgatgtgtggtatccgggcggtgtggtgagcagagtgccctttccaagtcctagttgtgtcaaaagcgagatgggctcttggatggagagctactccggaccctatggggacatgcgtttggagactgccagggaccatgttctacccattgactattactttccacctcagaagacctgtctgatctgcggtgatgaagcttctggctgtcactatggagctctcacttgtggaagctgcaaagtcttctttaaaagagccgctgaagggaaacagaagtacctgtgtgccagcagaaatgattgtaccatcgataaattccgaaggaaaaattgtccatcttgtcgcctccggaaatgctatgaagcagggatgactctgggagcccggaagctaaagaaactggggaatctgaaactgcaagaggaaggagaggcttccaatgtcaccagccccactgaggagccaacccagaagctgacggtgtcacacattgaaggctatgagtgtcagcccatctttctgaatgtccttgaagccatcgagccaggcgtggtgtgtgctggacatgacaacaaccagcccgactcctttgcagccttgctctctagccttaatgaattgggtgaaaggcagcttgtacatgtggtcaagtgggccaaggccttgccgggcttccgcaacctgcacgtggatgaccagatggcagtcattcagtactcctggatggggctcatggtgtttgccatgggctggcgatccttcaccaatgtcaactccaggatgctctacttcgcccctgacctggttttcaatgagtaccgcatgcacaagtcccggatgtacagccagtgtgtccgaatgaggcacctctctcaagaatttggatggctccaaatcaccccgcaggaatttttgtgcatgaaggcgctgctgctattcagcattattccagtggatgggctgaaaaatcaaaaattctttgatgaacttcgaatgaactacatcaaggaacttgatcgtatcattgcttgcaagagaaaaaatcccacatcctgctcaaggcgcttctaccagctcaccaagctcctggactctgtgcaacctattgctcgagagctgcatcagttcacttttgacctgctaatcaagtcccacatggtgagcgtggactttccagaaatgatggcagaaatcatctccgtgcaagtgcccaagattctttctgggaaagtcaagcccatctatttccacacgcagtgaagctttagaagcccctgtcttctcaccaccaccaccaccacccccgccccctactttttaaaaccttggtgtttgtttcatctgcttttagaacgtgcgatatttccagtacctacttttttgctgaatccaaagatatataaatataaatatatatattttataaagatcaaaatgatataaaggagatacgtttcttcctttaaaaaaaaacaaatagaaattcattgttaatgttcgtattatgatgccacttttctaaatcgtgcatcttgtatatgtgctgccgaagtgagcactaaatcgtgcatcttgatggaaaggtgtaaatatcaatcaccaggactgcttagttaattggtatttaatatctaggtgaattgggggtatggggtgggggtatgacaggacatacttggcttgccctcctcgaaatgactgccagttttccatatgcctgtccagttccaggaattttgttcacaagtgtgggggcgtcataggagtaaagcctgtaggacagatgaaaccccctgcttcagaggagcaagagctccaaggccaactgtttaacaagcaaggtgtttttttttctttctttctttctctttccaatgtgtggaattttgatgatggaaatgtcattctgagagcaatatttaaaattctcaggaattgacttatagtattgagaatgaattcagttgcaatcaagtttacattaaatgttgcttttaaaaaatctcagtcatacttcacactgcaaagaatccagatcacatgggataacttatgagatggttgagtggtaagctctgactgctttgctgatcattttgtaaataaaggtttc

SEQ ID NO: 2 - protein, 907 aa, Canine
MEVQLGLGRVYPRPPSKTYRGAFQNLFQSVREVIQNPGPRHPEAVSAAPPGAHLQQQQQQQQQQETSPRQQQQQQQGDDGSPQAQSRGPTGYLALDEEQQPSQQRSASKGHPESACVPEPGVTSATGKGLQQQQPAPPDENDSAAPSTLSLLGPTFPGLSSCSTDLKDILSEAGTMQLLQQQRQQQQQQQQQQQQQQQQQQQEVVSEGSSSGRAREAAGASTSSKDSYLGGSSTISDSAKELCKAVSVSMGLGVEALEHLSPGEQLRGDCMYAPLLGGPPAVRPCAPLAECKGSLLDDGPGKGTEETAEYSPFKAGYAKGLDGDSLGCSSSSEAGGSGTLEMPSTLSLYKSGALDEAAAYQSRDYYNFPLSLGGPPPHPPPPHPHTRIKLENPLDYGSAWAAAAAQCRYGDLASLHGAGAAGPSSGSPSATTSSSWHTLFTAEEGQLYGPCGGSGGGSAGDGGSVAPYGYTRPPQGLAGQEGDFPPPDVWYPGGVVSRVPFPSPSCVKSEMGSWMESYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASNVTSPTEEPTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

(19) United States
(12) Patent Application Publication
(10) Pub. No.: US 2002/0161194 A1
(43) Pub. Date: Oct. 31, 2002

(54) Cannine androgen receptor

(76) Inventors: Tessa A. Castleberry, Groton, CT (US); Bihong Lu, Killingworth, CT (US); Thomas A. Owen, East Lyme, CT (US); Steven L. Smock, Baltic, CT (US)

Correspondence Address: Gregg C. Benson, Pfizer Inc., Patent Department, MS 4159, Eastern Point Road, Groton, CT, 06340, US

(21) Appl. No.: 10/008,739

(22) Filed: Nov. 9, 2001

Related U.S. Application Data
(60) Provisional application No. 60/247,373, filed on Nov. 9, 2000.

(51) Int. Cl. C07K 1/00; C07K 14/00; C07K 17/00

(52) U.S. Cl. 530/350

(57) ABSTRACT

The present invention provides isolated canine AR protein, DNA molecules encoding said protein, vectors and cells carrying said DNA molecules, specific bind partners for the protein, and methods of discovering ligands for canine AR.

### CROSS REFERENCE TO RELATED APPLICATION

This application claims the benefit of U.S. Provisional Application No. 60/247,373 filed Nov. 9, 2000.

### FIELD OF THE INVENTION

The present invention relates to canine androgen receptor (canine AR or caAR herein) corresponding to that found in canine tissues. In other aspects, the invention covers, inter alia, structural variants of caAR, polynucleotides that encode caAR and its structural variants, expression vectors containing the polynucleotides, and cells transformed by the expression vectors.

### BACKGROUND OF THE INVENTION

Sex steroids, including testosterone, play a major role in determining peak bone mass and the subsequent loss of total bone mass with increasing age. It is well established in animal models that testosterone deficiency, due to orchidectomy, results in the loss of bone from the male skeleton and that the administration of exogenous androgens can either prevent this loss or restore the lost bone mass. In human males, bone mass and levels of circulating androgens both decrease with age. Some human clinical studies have suggested that androgen supplementation of the elderly can have beneficial effects with respect to the skeleton, although more comprehensive, longitudinal studies need to be performed in order for definitive conclusions to be reached.

Testosterone and its active metabolite dihydrotestosterone (DHT) bind with high affinity to the androgen receptor (AR), a member of the nuclear hormone receptor superfamily. Members of this receptor superfamily function as transcription factors by binding in concert with accessory proteins to specific DNA response elements in the promoters of androgen responsive genes, thereby modulating their expression. Molecular cloning of the cDNAs encoding the AR from diverse species has revealed a very highly conserved molecule with its various functions divided into discrete modular domains. The domain responsible for transactivation of androgen responsive genes lies within the N-terminal half of the AR. The two zinc fingers comprising the DNA binding domain reside within 68 amino acids in the middle of the molecule, while the ligand binding domain occupies approximately the C-terminal 30% of the AR.

### SUMMARY OF THE INVENTION

To further the understanding of the molecular and biochemical actions of androgenic hormones and related compounds throughout the canine animal, as well as to expand the understanding of androgen receptors, we have cloned, expressed, and characterized the canine AR receptor. The canine AR receptor has functional characteristics in common with the previously reported AR receptors from human and other mammals.

Thus, a canine cDNA has been isolated that encodes an open reading frame with a DNA and predicted protein sequence that is highly homologous to the reported AR receptor from human and other species. When this canine cDNA is expressed in A293S cells, it encodes a protein with ligand binding properties characteristic of AR and, when activated by appropriate ligands, results in the transcriptional activation of androgen responsive transcriptional elements within cells. The cloning of canine AR will aid in furthering the understanding of the molecular and biochemical actions of androgens in canine bone.

The present invention has several aspects. In a first aspect, the present invention relates to isolated proteinaceous molecules having an activity of caAR and comprising an amino acid sequence corresponding to SEQ ID NO:2.

In another aspect, the present invention relates to isolated DNA molecules encoding the proteinaceous molecules described above (see, for example, SEQ ID NO:1).

Other aspects of the present invention relate to recombinant expression vectors capable of transferring the recited polynucleotide molecules to suitable host cells, and to cells transformed by these expression vectors.

In another aspect the present invention relates to methods of producing the recited proteinaceous molecules by recombinant means.

In a further aspect of the present invention, it relates to pharmaceutical compositions comprising the recited proteinaceous molecules.

In yet another aspect, the present invention relates to specific binding partners to the recited proteinaceous molecules.

In still another aspect, the present invention relates to methods of discovering ligands for canine AR.

The above aspects of the present invention enable production of large quantities of proteinaceous molecules having canine AR activity. It is contemplated that such molecules can be used, e.g., for veterinary purposes to treat dogs, as described in greater detail herein.

### DESCRIPTION OF THE FIGURE SHEETS

FIG. 1 provides the nucleotide and predicted protein sequence of the canine androgen receptor. Screening of a canine kidney cDNA library using a human AR probe together with PCR based cloning of the complete protein coding region resulted in this composite of the canine AR sequence (2721 bp protein coding region plus 856 bp of 3′-untranslated sequence). The predicted amino acid sequence of the open reading frame starting at the initiation methionine at nucleotide 1 is shown in single letter format below the DNA sequence. The solid black bar underlines the peptide recognized by the anti-human AR antibody. The nucleotide sequence of the canine AR cDNA has been deposited in GenBank with the accession number AF197950.

FIG. 2 provides a comparison of the DNA sequences of the protein coding region of canine and human androgen receptors. The DNA sequences of the protein coding regions of the canine and human androgen receptors are compared. Boxed regions indicate areas of 100% nucleotide identity between the two species. Gaps have been introduced where necessary for alignment. This analysis reveals a molecule with a high degree of overall conservation, especially within the DNA binding (DBD) and ligand binding domains(LBD).

FIG. 3 provides a comparison of the predicted protein sequences of canine, human, chimpanzee, macaque, lemur, rat, and mouse androgen receptors. Residues conserved among all seven species are boxed. Gaps have been introduced where necessary for alignment. The arrow at amino acid 545 of the canine protein indicates the start of the absolutely conserved DNA binding domain (DBD, residues 545-612), while the arrow at amino acid 655 of the canine protein sequence marks the beginning of the absolutely conserved ligand binding domain (LBD, residues 655-end).

FIG. 4 provides a graphical characterization of ligand binding to A293S cells expressing the canine androgen receptor. Competitive binding of various ligands with ³H-DHT for AR in extracts from A293S cells transiently transfected with the canine AR cDNA. The indicated concentrations of DHT (▪), testosterone (●), stanozolol (▴) or dexamethasone (♦) were incubated with 10 μl cell extract for 1 hr at 4° C. in the presence of 1 nM ³H-DHT. Specific binding was determined by scintillation counting following recovery of the receptor by filtration through hydroxylapatite. Error bars represent one standard deviation. These studies demonstrated specific displacement of ³H-DHT by DHT, testosterone, and the anabolic steroid stanozolol, with IC50 values of 1.3 nM, 2.5 nM and 3.8 nM, respectively. Binding of ³H-DHT was weakly displaced by dexamethasone, a ligand for the glucocorticoid receptor, with an IC50 of 6 μM.

FIG. 5 provides a bar graph showing the induction of ARE-Luciferase activity by DHT in A293S cells expressing the canine androgen receptor. A293S cells were transiently transfected with 5 μg canine AR/pcDNA3.1(+), 2 μg ARE-Luciferase, 1 μg pEGFP and 1 μg □-gal per dish using Lipofectamine. Control plates were transfected with all plasmids except canine AR. 24 hr after transfection, cells were split into 12-well dishes and, 24 hours later, were treated with either vehicle (0.1% ethanol) or DHT (10⁻⁷ M) for an additional 24 hr, harvested, and analyzed for luciferase and β-galactosidase activities. Luciferase activity was normalized for transfection efficiency using β-galactosidase activity. Error bars represent one standard deviation. There was no induction of luciferase activity by DHT in cells lacking the canine AR while there was a 6-fold induction of luciferase activity by DHT in cells transfected with canine AR.

### DETAILED DESCRIPTION OF THE INVENTION

Sex steroids, including testosterone, play a major role in determining peak bone mass and the subsequent loss of total bone mass with age. Testosterone and its active metabolite dihydrotestosterone (DHT) bind with high affinity to the androgen receptor (AR), a member of the nuclear hormone receptor superfamily. These receptors function as transcription factors, binding, together with accessory proteins, to specific DNA response elements in the promoters of androgen responsive genes. Cloning of the cDNAs encoding the AR from several species has revealed a highly conserved, modular molecule with transactivation, DNA binding, and ligand binding domains. To further study AR function in a model species of relevance to bone, we cloned the canine AR by first screening a canine kidney cDNA library and then by cloning the remaining 5′segment by PCR from canine ventral prostate cDNA. The complete sequence obtained was 3577 bp. This sequence contained a single open reading frame of 2721 bp, potentially encoding a protein of 907 amino acids with a predicted molecular weight of 98.7 kD. Sequence analysis of the protein encoded by this open reading frame reveals that the modular domains providing the DNA binding and ligand binding functions are identical to those reported for the human, mouse, and rat ARs. Northern analysis of poly-A⁺ RNA from ventral prostate revealed three very low abundance transcripts of approximately 9 kb. RT-PCR analysis of canine ventral prostate, spleen, skeletal muscle, heart, testis, liver, and kidney demonstrated a rather ubiquitous pattern of expression. Competition binding studies using ³H-DHT as ligand demonstrated specific displacement by DHT, testosterone, and the anabolic steroid stanozolol, with IC50 values of 1.3 nM, 2.5 nM, and 3.8 nM, respectively. Binding of ³H-DHT was weakly displaced by dexamethasone, a ligand for the glucocorticoid receptor, with an IC50 of 6 μM. Binding of DHT also resulted in the stimulation of an ARE-luciferase reporter following cotransfection with the canine AR into A293S cells. Immunohistochemistry using an antibody directed to the C-terminal 20 amino acids showed strong staining of the secretory epithelial cells in canine ventral prostate and in the periosteal layer as well as in active osteoblasts at several sites. Together, these data indicate that we have cloned the canine androgen receptor and that its functional DNA binding and ligand binding domains are absolutely conserved with those found in the human AR.

Definitions

“Proteinaceous molecule” generally encompasses any molecule made up of a plurality of amino acids. The term is broad enough to include peptides, oligopeptides, and proteins. Typically, the amino acids in the proteinaceous material will be selected from the 20 naturally occurring amino acids. However, amino acid analogs and derivatives could also be included in the proteinaceous molecule. The proteinaceous molecule will usually be made up of about the same number of amino acids contained in naturally occurring caAR (i.e., about 907 amino acids). In addition, however, the proteinaceous molecule may have a greater or lesser number of amino acids, as long as the molecules retain an activity of caAR. This activity need not be quantitatively the same as the activity of the natural protein, and may be more or less than that activity, as long as it is measurable by an assay of caAR activity (see below). Preferably, such molecules will possess at least about 50% of the activity of the naturally occurring caAR. In some hosts, expression of recombinant caAR will result in a protein having an N-terminal methionine residue. Amino acid sequences containing such a residue on the N-terminus thereof are also within the scope of the present invention.

By “conservative substitution” is meant a substitution, addition, or deletion of an amino acid in a proteinaceous molecule that is not expected to significantly affect the activity of thereof. For example, the replacement of one hydrophobic amino acid for another in a transmembrane region of a proteinaceous molecule will seldom have any significant impact on the activity of thereof. Other conservative substitutions are well known to those skilled in the art.

By “activity of caAR” is meant any activity that is measurable by an in vivo or in vitro caAR assay. Qualitatively, the activity will generally be one that is possessed by the naturally occurring caAR protein. Cells expressing, or thought to be expressing, caAR protein may be assayed for both the levels of caAR receptor activity and levels of caAR protein. Assessing caAR receptor activity preferably involves the direct introduction of a labeled ligand to the cells and determining the amount of specific binding of the ligand to the caAR-expressing cells. Binding assays for receptor activity are known in the art (Liao et al., J. Steroid Biochem., 20:11-17 (1984)). Levels of caAR protein in host cells may be quantitated by a variety of techniques including, but not limited to, immunoaffinity and/or ligand affinity techniques. caAR-specific affinity beads or caAR-specific antibodies are used to isolate ³⁵S-methionine labeled or unlabelled caAR protein. Labeled caAR protein may be analyzed by SDS-PAGE. Unlabelled caAR protein may be detected by Western blotting, ELISA, or RIA assays employing caAR-specific antibodies.

By “isolated” is meant that a substance is substantially free from normally occurring impurities or other molecules, especially other proteinaceous molecules, salts, other cellular constituents, and the like. Isolation may typically be carried out by standard methods in the art of nucleotide, peptide, and/or protein purification or synthesis. Following expression of caAR in a host cell, caAR protein may be recovered to provide caAR in active form, capable of binding caAR-specific ligands. Several caAR purification procedures are available and suitable for use. Recombinant caAR may be purified from cell lysates and extracts, or from conditioned culture medium, by various combinations of, or individual application of salt fractionation, ion exchange chromatography, size exclusion chromatography, hydroxylapatite adsorption chromatography, and hydrophobic interaction chromatography. In addition, recombinant caAR can be separated from other cellular proteins by use of an immunoaffinity column made with monoclonal or polyclonal antibodies specific for full length nascent caAR, or polypeptide fragments of caAR.

“Polynucleotide sequences” encompass both DNA- and RNA-containing molecules. The DNA molecules will preferably be intronless sequences (i.e., cDNA), but can contain enhancer sequences, termination sequences, and the like, to facilitate or increase expression in a particular host. mRNA molecules are also compassed by the term.

By “recombinant expression vector” is meant a vector (e.g., a plasmid or λphage) that is capable of transferring polynucleotide sequences contained therein into cells of a host organism for expression of the transferred sequences. The sequences are operably linked to other sequences capable of effecting or modulating their expression. Such expression vectors must be replicable in a host organism. For example, any DNA sequence that is capable of effecting expression of a specified DNA sequence disposed therein is included in this term as it is applied to the specified sequence.

The “cells” that may be transformed by way of the vectors described above are those that are capable of expressing the polynucleotide sequences that have been transferred by the vector. Culturing conditions for such cells may be those standard in the art of recombinant protein production.

“Specific binding partners” are molecules that are capable, on a molecular level, of recognizing and interacting with the proteinaceous or polynucleotide molecules described herein. Included within this term are immunological binding partners such as antibody molecules, antigen-binding fragments of antibodies (e.g., Fab and F(ab′)₂ fragments), single chain antigen-binding molecules, and the like, whether produced by hybridoma or rDNA technology. Other proteinaceous or non-proteinaceous binding partners are also included within the broad term.

Proteinaceous Molecules

The proteinaceous molecules of this invention will now be described in greater detail. SEQ ID NO:2 corresponds to a naturally occurring canine peptide having 907 amino acids. However, the androgen receptor in humans is known to have a “poly-Q” region, a region within the protein that consists of a variable number of repetitive glutamine amino acid units. What appears to be a similar poly-Q region is present in the canine sequence as well (see amino acid numbers 180-202). In humans, this region varies, consisting of from about 7-36 consecutive glutamine residues. While it is presently not known to what extent this region might vary in canines, it is reasonably assumed that a similar level of variation does exist. Thus, the present invention, when it refers to the amino acid or nucleic acid sequence of the canine AR, is intended to encompass all naturally occurring levels of variation in this poly-Q region. See Edwards et al., Int. J. Cancer (Pred. Oncol.), 84:458-65 (1999); and Irvine et al., Human Molec. Genetics, 9(2):267-74 (2000).

The family of caAR proteins provided herein also includes proteinaceous molecules in which one or more of the amino acids in the above-recited amino acid sequences has been deleted, modified, or changed to another amino acid. Site directed mutagenesis is a preferred technique enabling conversion of one amino acid to another. For example, one or more of the cysteine residues may be changed to another amino acid such as serine. One possible reason for such a change would be to eliminate one or more unwanted disulfide bonds. See, for example, U.S. Pat. No. 4,518,584.

Other contemplated specific changes in the natural amino acid sequences involve modification of any asparagine glycosylation sites. Modification of asparagine can eliminate glycosylation at the modified site. Thus, for example, the asparagine could be changed to a glutamine, thereby eliminating glycosylation at the site. See, for example, Miyajima et al., EMBO J., 5(6):1993 (1986).

To determine the caAR cDNA sequence(s) that yields optimal levels of receptor activity and/or caAR protein, caAR cDNA molecules including but not limited to the following can be constructed: the full-length open reading frame of the caAR cDNA and various constructs containing portions of the cDNA encoding only specific domains of the receptor protein or rearranged domains of the protein. All constructs can be designed to contain none, all, or portions of the 5′ and/or 3′ untranslated region of caAR cDNA. caAR activity and levels of protein expression can be determined following the introduction, both singly and in combination, of these constructs into appropriate host cells. Following determination of the caAR cDNA cassette yielding optimal expression in transient assays, this caAR cDNA construct is transferred to a variety of expression vectors (including recombinant viruses), including but not limited to those for mammalian cells, plant cells, insect cells, oocytes, E. coli, and yeast cells.

The modifications of the amino acid sequences described above that form a part of the present invention are those that result in proteinaceous molecules having an activity of canine AR. Such activity may be determined using the assays described herein, or equivalent assays now known or yet to be developed. The family of proteinaceous molecules of the present invention will also generally be highly homologous to SEQ ID NO:2, which means that several of the amino acids in each sequence may be deleted, modified, or changed (assuming the resulting proteinaceous material retains at least some activity in common with canine AR).

The proteinaceous molecules of the present invention may further be labeled by attachment to a detectable marker substance (e.g., radiolabeled with ¹²⁵I) to provide reagents useful in vitro or in vivo.

Polynucleotide Sequences

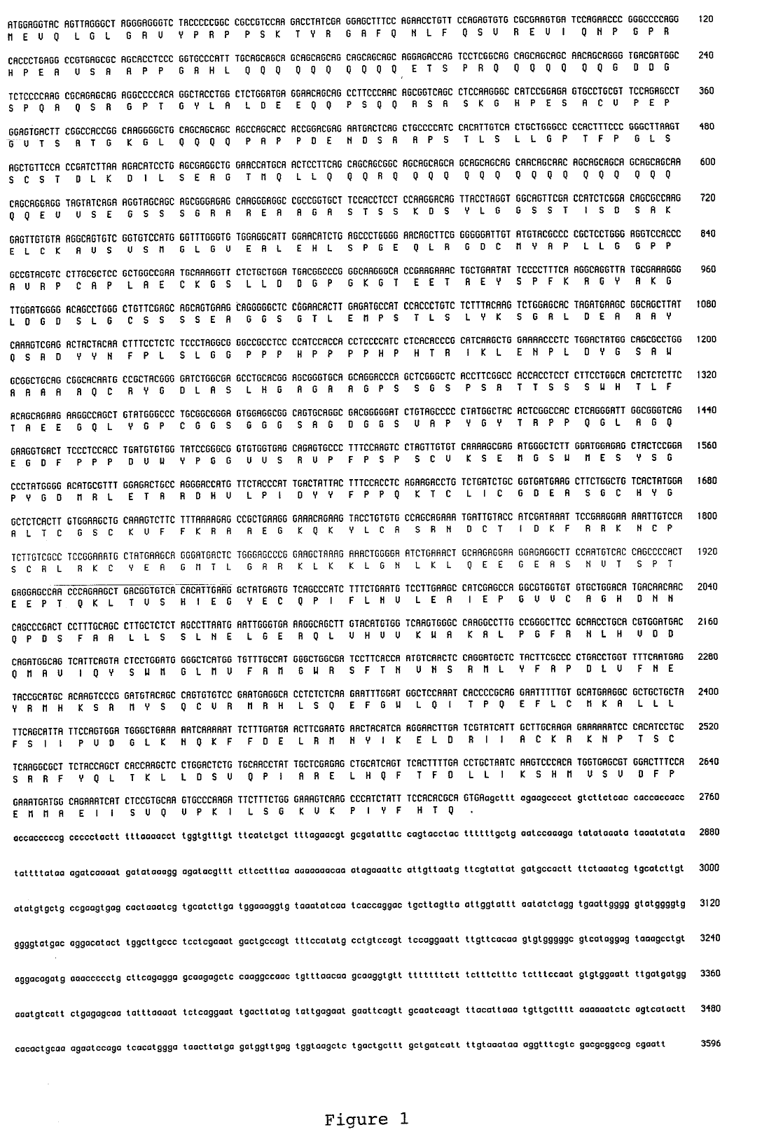

The present invention is also directed to polynucleotide sequences. Preferred polynucleotide sequences are DNA molecules that encode the proteinaceous molecules of the present invention, described above. A preferred DNA sequence (SEQ ID NO:1) is the one shown in FIG. 1 herein. The novel cDNA sequence illustrated in FIG. 1 included in a plasmid has been deposited before filing date in GenBank under accession number AF197950. The cDNA of FIG. 1 is described in greater detail herein.

It is to be recognized that more than one DNA sequence can encode the same amino acid sequence, due to the degeneracy of the genetic code. All such sequences are encompassed by the polynucleotide sequences described herein.

The presently preferred method of obtaining the cDNA of FIG. 1 is described herein. However, any of a variety of alternative procedures may be used to clone caAR cDNA. These methods include, but are not limited to, direct functional expression of the caAR cDNA following the construction of an caAR-containing cDNA library in an appropriate expression vector system. Another method is to screen an caAR-containing cDNA library constructed in a bacteriophage or plasmid shuttle vector with a labeled oligonucleotide probe designed from the amino acid sequence of the caAR protein. The preferred method consists of screening an caAR-containing cDNA library constructed in a bacteriophage or plasmid shuttle vector with a partial cDNA encoding the caAR protein. This partial cDNA is obtained by the specific PCR amplification of caAR DNA fragments through the design of degenerate oligonucleotide primers from the amino acid sequence known for other G protein-coupled receptors which are related to the caAR receptors.

It is readily apparent to those skilled in the art that other types of libraries, as well as libraries constructed from other cells or cell types, may be useful for isolating caAR-encoding DNA. Other types of libraries include, but are not limited to, cDNA libraries derived from other cells or cell lines other than canine kidney cells, and genomic DNA libraries.

It is readily apparent to those skilled in the art that suitable cDNA libraries may be prepared from cells or cell lines that have caAR activity. The selection of cells or cell lines for use in preparing a cDNA library to isolate caAR cDNA may be done by first measuring cell associated caAR activity using the known labeled ligand binding assay cited herein.

Preparation of cDNA libraries can be performed by standard techniques well known in the art. Well known cDNA library construction techniques can be found for example, in Maniatis et al., Molecular Cloning: A Laboratory Manual (Cold Spring Harbor Laboratory, Cold Spring Harbor, N.Y., 1982).

It is also readily apparent to those skilled in the art that DNA encoding caAR may also be isolated from a suitable genomicDNA library. Construction of genomic DNA libraries can be performed by standard techniques well known in the art. Well known genomic DNA library construction techniques can be found in Maniatis et al., Molecular Cloning: A Laboratory Manual (Cold Spring Harbor Laboratory, Cold Spring Harbor, N.Y., 1982).

In order to clone the caAR gene by one of the preferred methods, the amino acid sequence or DNA sequence of caAR or a homologous protein is necessary. To accomplish this, caAR protein or a homologous protein may be purified and partial amino acid sequence determined by automated sequenators. It is not necessary to determine the entire amino acid sequence, but the linear sequence of two regions of 6 to 8 amino acids can be determined for the PCR amplification of a partial caAR DNA fragment.

Once suitable amino acid sequences have been identified, the DNA sequences capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the caAR sequence but others in the set may be capable of hybridizing to caAR DNA even though they contain mismatches. The mismatched DNA oligonucleotides may still sufficiently hybridize to the caAR DNA to permit identification and isolation of caAR encoding DNA.

Using one of the preferred methods, cDNA clones encoding caAR are isolated in a two-stage approach employing polymerase chain reaction (PCR) based technology and cDNA library screening. In the first stage, NH₂-terminal and internal amino acid sequence information from the purified caAR or a homologous protein is used to design degenerate oligonucleotide primers for the amplification of caAR-specific DNA fragments. In the second stage, these fragments are cloned to serve as probes for the isolation of full length cDNA from a cDNA library derived from canine kidney cells.

Polynucleotide products of the present invention may be labeled with detectable markers (such as radiolabels and non-isotopic labels such as biotin) and employed, for example, in DNA hybridization processes to locate the canine gene position and/or the position of any related gene family in a chromosomal map. They may also be used for identifying canine gene disorders at the DNA level and used as gene markers for identifying neighboring genes and their disorders.

Expression Vectors, Hosts, and Recombinant Methods

The cloned caAR cDNA obtained through the methods described herein may be recombinantly expressed by molecular cloning into an expression vector containing a suitable promoter and other appropriate transcription regulatory elements, and transferred into prokaryotic or eukaryotic host cells to produce recombinant caAR. Techniques for such manipulations can be found described in Maniatis et al., supra, and are well known in the art.

Expression vectors are defined herein as DNA sequences that are required for the transcription of cloned DNA and the translation of their mRNAs in an appropriate host. Such vectors can be used to express eukaryotic DNA in a variety of hosts such as bacteria, blue-green algae, plant cells, insect cells, and animal cells.

Specifically designed vectors allow the shuttling of DNA between hosts such as bacteria-yeast or bacteria-animal cells. An appropriately constructed expression vector preferably contains: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency. Expression vectors may include, but are not limited to, cloning vectors, modified cloning vectors, specifically designed plasmids, or viruses.

A variety of mammalian expression vectors may be used to express recombinant caAR in mammalian cells. Commercially available mammalian expression vectors that may be suitable for recombinant caAR expression, include but are not limited to, pMClneo (Stratagene), pXT1 (Stratagene), pSG5 (Stratagene), pcDNAI, pcDNAlamp (Invitrogen), EBO-pSV2-neo (ATCC 37593) pBPV-1(8-2) (ATCC 37110), pdBPV-MMTneo (342-12) (ATCC 37224) pRS-Vgpt (ATCC 37199), pRSVneo (ATCC 37198), pSV2-dhfr (ATCC 37146), pUCTag (ATCC 37460), and IZD35 (ATCC 37565)

DNA encoding caAR may also be cloned into an expression vector for expression in a host cell. Host cells may be prokaryotic or eukaryotic, including but not limited to bacteria, yeast, mammalian cells including but not limited to cell lines of human, bovine, porcine, monkey, and rodent origin, and insect cells including but not limited to drosophila derived cell lines. Cell lines derived from mammalian species which may be suitable and which are commercially available, include but are not limited to, CV-1 (ATCC CCL 70), COS-1 (ATCC CRL 1650), COS-7 (ATCC CRL 1651), CHO-KI (ATCC CCL 61), 3T3 (ATCC CCL 92), NIHi3T3 (ATCC CRL 1658), HeLa (ATCC CCL 2), Cl271 (ATCC CRL 1616), BS-C-1 (ATCC CCL 26), and MRC-5 (ATCC CCL 171).

The expression vector may be introduced into host cells via any one of a number of techniques including but not limited to transformation, transfection, protoplast fusion, and electroporation. The expression vector-containing cells are individually analyzed to determine whether they produce caAR protein. Identification of caAR expressing cells may be done by several means, including but not limited to immunological reactivity with anti-caAR antibodies, and the presence of host cell-associated caAR activity.

Expression of caAR DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

A variety of prokaryotic cells known to those of ordinary skill in this art may be utilized. A few exemplary strains include E. coli, Bacillus subtilis, and various strains of Pseudomonas.

In addition to bacteria, eukaryotic microbes, such as yeast, may also be used as a host. Laboratory strains of Saccharomyces cerevisiae are most commonly used.

It is also possible to express genes encoding polypeptides in eukaryotic host cell cultures derived from multi-cellular organisms. Useful host cell lines include Vero, HeLa, COS, and Chinese hamster ovary (CHO) cells. Expression vectors for such cells ordinarily include promoters and control sequences compatible with mammalian cells such as, for example, the commonly used early and later promoters from Simian Virus 40 (SV40) or other viral promoters such as those derived from polyoma, adenovirus 2, bovine papilloma virus, avian sarcoma viruses, immunoglobulin promoters, and heat shock promoters. Enhancer regions may also be included as desired.

Other examples of hosts, vectors, enhancers, promoters, etc., may be found in the following exemplary U.S. Pat. Nos. 4,810,643; 4,766,075; and 4,847,201, each of which is incorporated by reference herein.

Specific Binding Partners

Specific binding partners directed to the proteinaceous molecules and polypeptides of the present invention may be generated by any standard technique known to those of skill in the art. Preferred specific binding partners are immunological binding partners such as intact antibodies and fragments thereof. The immunological binding partners are preferably monoclonal antibodies directed to a specific antigen, which are prepared by standard techniques monoclonal antibody production.

These specific binding partners may be utilized, for example, to purify the proteinaceous materials or polynucleotides of the present invention. Specific binding partners in labeled form may be utilized to indicate the presence of the proteinaceous molecules or polynucleotides of the present invention. In one preferred embodiment, a specific binding partner of a polynucleotide of the present invention may be utilized in labeled form to locate the natural gene on a chromosome.

Monospecific antibodies to caAR are purified from mammalian antisera containing antibodies reactive against caAR or are prepared as monoclonal antibodies reactive with caAR using the technique of Kohler and Milstein, Nature, 256:495-497 (1975). Monospecific antibody as used herein is defined as a single antibody species or multiple antibody species with homogenous binding characteristics for caAR. Homogenous binding as used herein refers to the ability of the antibody species to bind to a specific antigen or epitope, such as those associated with the caAR, as described above. caAR specific antibodies are raised by immunizing animals such as mice, rats, guinea pigs, rabbits, goats, horses, and the like, with an appropriate concentration of caAR either with or without an immune adjuvant.

Preimmune serum is collected prior to the first immunization. Each animal receives between about 0.1 μg and about 1000 μg of caAR associated with an acceptable immune adjuvant. Such acceptable adjuvants include, but are not limited to, Freund's complete, Freund's incomplete, alum-precipitate, water in oil emulsion containing Corynebacterium parvum, and tRNA. The initial immunization consists of the enzyme in, preferably, Freund's complete adjuvant at multiple sites either subcutaneously (SC), intraperitoneally (IP), or both. Each animal is bled at regular intervals, preferably weekly, to determine antibody titer. The animals may or may not receive booster injections following the initial immunization. Those animals receiving booster injections are generally given an equal amount of caAR in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20° C.

Monoclonal antibodies (mAb) reactive with caAR are prepared by immunizing inbred mice, preferably Balb/c, with caAR. The mice are immunized by the IP or SC route with about 1 μg to about 100 μg, preferably about 10 μg, of caAR in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 1 to about 100 μg of caAR in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1, MPC-11, S-194, and Sp 2/0, with Sp 2/0 being preferred. The antibody producing cells and myeloma cells are fused in polyethylene glycol, about 1000 mol. wt., at concentrations from about 30% to about 50%. Fused hybridoma cells are selected by growth in hypoxanthine, thymidine, and aminopterin supplemented Dulbecco's Modified Eagles Medium (DMEM) by procedures known in the art. Supernatant fluids are collected from growth positive wells on about days 14, 18, and 21 and are screened for antibody production by an immunoassay such as solid phase immunoradioassay (SPIRA) using caAR as the antigen. The culture fluids are also tested in the Ouchterlony precipitation assay to determine the isotype of the mAb. Hybridoma cells from antibody positive wells are cloned by a technique such as the soft agar technique of MacPherson, (Soft Agar Techniques, in Tissue Culture Methods and Applications, IQ-use and Paterson, Eds., Academic Press, 1973).

Monoclonal antibodies are produced in vivo by injection of pristine primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production of anti-caAR mAb is carried out by growing the hydridoma in DMEM containing about 2% fetal calf serum to obtain sufficient quantities of the specific mAb. The mAb are purified by techniques known in the art.

Antibody titers of ascites or hybridoma culture fluids are determined by various serological or immunological assays which include, but are not limited to, precipitation, passive agglutination, enzyme-linked immunosorbent antibody (ELISA) technique, and radioimmunoassay (RIA) techniques. Similar assays are used to detect the presence of caAR in body fluids or tissue and cell extracts.

It is readily apparent to those skilled in the art that the above described methods for producing monospecific antibodies may be utilized to produce antibodies specific for caAR polypeptide fragments or full-length caAR polypeptide.

caAR antibody affinity columns are made by adding the antibodies to Affigel-10 (Biorad), a gel support that is pre-activated with N-hydroxysuccinimide esters such that the antibodies form covalent linkages with the agarose gel bead support. The antibodies are then coupled to the gel via amide bonds with the spacer arm. The remaining activated esters are then quenched with 1M ethanolamine HCl (pH 8). The column is washed with water followed by 0.23M glycine HCl (pH 2.6) to remove any non-conjugated antibody or extraneous protein. The column is then equilibrated in phosphate buffered saline (pH 7.3) and the cell culture supernatants or cell extracts containing caAR or caAR fragments are slowly passed through the column. The column is then washed with phosphate buffered saline until the optical density (A₂₈₀) falls to background, then the protein is eluted with 0.23M glycine-HCl (pH 2.6). The purified caAR protein is then dialyzed against phosphate buffered saline.

Screening For Androgen Receptor Antagonists And Agonists

The polypeptides and antibodies of the invention and other compounds may be screened for competition with androgenic compounds and for antagonistic or agonistic properties using assays described herein.

In one example, those antibodies that recognize the AR on the intact cells are screened for their ability to compete with testosterone and other androgenic hormones, and for binding to the AR. Cells expressing AR on the cell surface are incubated with the ¹²⁵I analogs of androgenic hormones in the presence or absence of the polyclonal or monoclonal antibody to be tested, for 4 h at 15° C. The antibody used may be from crude antiserum, cell medium, or ascites, or in purified form. After incubation, the cells are rinsed with binding buffer (e.g., physiological saline), lysed, and quantitatively analyzed for radioactivity using a gamma-counter. Antibodies that reduce binding of the androgenic hormone to the AR are classified as competitive; those which do not are noncompetitive.

The canine AR receptor of the present invention may be obtained from both native and recombinant sources for use in an assay procedure to identify receptor modulators. In general, an assay procedure to identify canine AR receptor modulators will contain the canine AR receptor of the present invention, and a test compound or sample which contains a putative canine AR receptor modulator. The test compounds or samples may be tested directly on, for example, purified receptor protein whether native or recombinant, subcellular fractions of receptor-producing cells whether native or recombinant, and/or whole cells expressing the receptor whether native or recombinant. The test compound or sample may be added to the receptor in the presence or absence of a known labeled or unlabelled receptor ligand. The modulating activity of the test compound or sample may be determined by, for example, analyzing the ability of the test compound or sample to bind to the receptor, activate the receptor, inhibit receptor activity, inhibit or enhance the binding of other compounds to the receptor, modifying receptor regulation, or modifying an intracellular activity.

The identification of modulators of caAR receptor activity are useful in treating disease states involving the caAR receptor activity. Other compounds may be useful for stimulating or inhibiting activity of the receptor. The isolation and purification of an caAR-encoding DNA molecule would be useful for establishing the tissue distribution of caAR receptors as well as establishing a process for identifying compounds which modulate caAR receptor activity.

Uses

The polypeptides, antibodies, and other compounds of the invention are useful for the diagnosis, classification, prognosis, and/or treatment of canine disorders which may be characterized as related to the interaction between a cell receptor of the invention and its specific ligand. For example, modulators of androgen receptor activity might be useful in treating cancers of the prostate or testicles. They might also be useful for treating frailty in canines, particularly frailty caused by muscular degeneration associated with disease or aging. However, frailty associated with bone loss might also be treatable by such methods. Additionally, canine mood and temper might be improved by treatment with modulators of caAR.

In a first example, the compounds of the invention are used to manufacture diagnostic agents which are used as canine diagnostic tools to diagnose risk of prostate cancer. It is known in humans that certain variations in the length of the poly-Q region of the AR are associated with increased risk of prostate cancer. It is quite possible that such variations in the length of the poly-Q region in the canine AR will also be associated with increased risk of prostate cancer.

Nucleic acids of the invention which encode an AR may also be linked to a selected tissue-specific promoter and/or enhancer and the resultant hybrid gene introduced, by standard methods (e.g., as described by Leder et al., U.S. Pat. No. 4,736,866, herein incorporated by reference), into an animal embryo at an early developmental stage (e.g., the fertilized oocyte stage), to produce a transgenic animal which expresses elevated levels of AR in selected tissues (e.g., the osteocalcin promoter for bone). Such promoters are used to direct tissue-specific expression of the AR in the transgenic animal. The form of AR utilized can be one which encodes an AR similar to that of the animal species used, or it can encode the AR homolog of a different species.

The invention now being generally described, the same will be better understood by reference to certain specific examples which are included herein for illustrative purposes only and are not intended to be limiting of the present invention, except where so stated.

### EXAMPLES

### Example 1

### Isolation of Total and Poly-A⁺ RNA

Canine tissues for RNA isolation were obtained from Comparative Medicine and Biology Support, Pfizer Central Research, in accordance with institutional policies and immediately frozen in liquid nitrogen. Total cellular RNA was isolated from these frozen canine tissues by centrifugation (100,000× g overnight) through a 5.7 M CsCl cushion following homogenization in 5 M guanidinium isothiocyanate. Total RNA was quantitated by absorbence at 260 nm and poly-A⁺ RNA was enriched using the poly(A) Quik mRNA isolation kit (Stratagene, La Jolla, Calif.).

### Example 2

### cDNA Library Construction and Screening

A cDNA library in λ TripIEx was constructed from canine kidney RNA by the custom services group of Clontech, Inc. (Palo Alto, Calif.). This library was screened by hybridization using a ³²P-labeled DNA fragment (Prime-It RmT Kit; Stratagene) corresponding to the C-terminal 242 amino acids of the human androgen receptor as probe. Filters were washed 3 times (20 minutes per wash) in 6× SSC/0.1% SDS at room temperature and 1 time (20 minutes) in 6× SSC/0.1% SDS at 55° C. Following conversion of 5 positive □TripIEx clones to pTripIEx, plasmid DNAs were prepared using PerfectPrep (5′→3′, Boulder, Colo.), cDNA insert sizes were determined by restriction digestion, and the clone with the largest insert (9C1) was sequenced using yeast AT-2 transposon insertion (Primer Island Kit, PE Applied Biosystems, Foster City, Calif.) and dideoxy sequencing methodologies. See FIG. 1.

### Example 3

### Cloning the Complete Canine Androgen Receptor Protein Coding Region

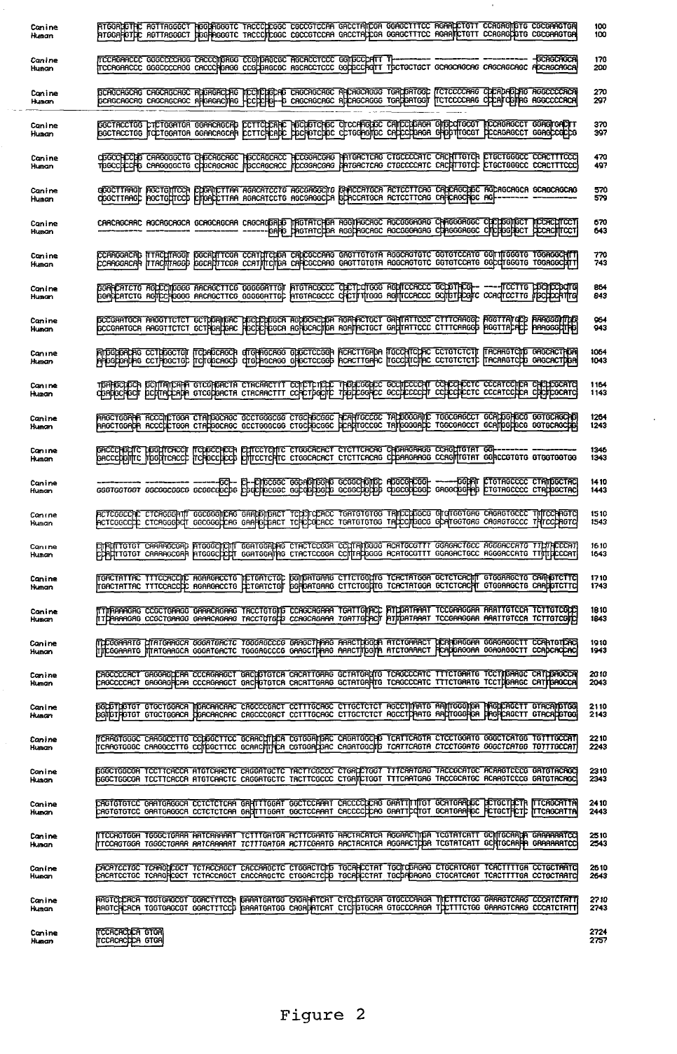

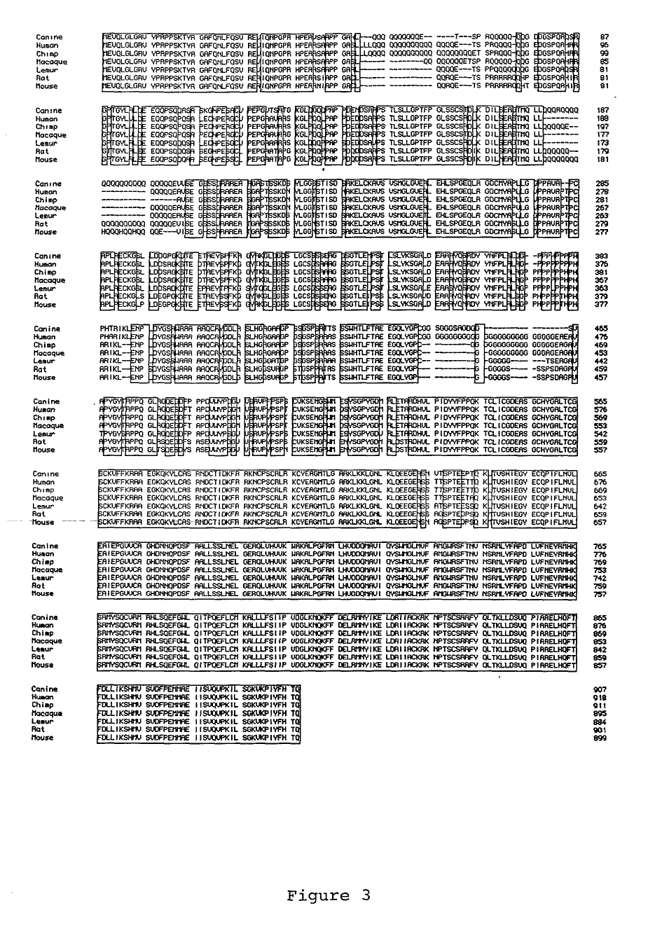

One □I canine ventral prostate poly-A⁺ RNA was reverse transcribed in a 20 □I reaction (15 min, 42° C.) using random primers and MuLV reverse transcriptase (PE Applied Biosystems). Five □I of reverse transcription reaction was amplified by PCR using a 5′ primer based on a consensus of the sequences of the human, mouse, and rat AR just upstream of the start of translation (5′-GTGGGCAGCTAGCTGCAG) and 3′ primer just downstream of the translation stop, based on the sequence of canine AR clone 9C1, obtained by cDNA library screening (5′-GGTGGTGGTGAGAAGACAGG). Amplification was performed for 33 cycles using the TaKaRa LA PCR kit (PanVera, Madison, Wis.), the resulting PCR product was subcloned into pCR2.1-TOPO (Invitrogen, Carlsbad, Calif.), and plasmid DNAs were prepared using PerfectPrep. Insert sizes were determined by restriction digestion and DNA sequence was obtained using dideoxy sequencing methodologies using 16 primer sets based on the sequence of canine clone 9C1 and consensus sequences based on the reported sequences of the human, mouse, and rat ARs. See FIGS. 2 and 3.

### Example 4

### Analysis of Binding to Canine Androgen Receptor

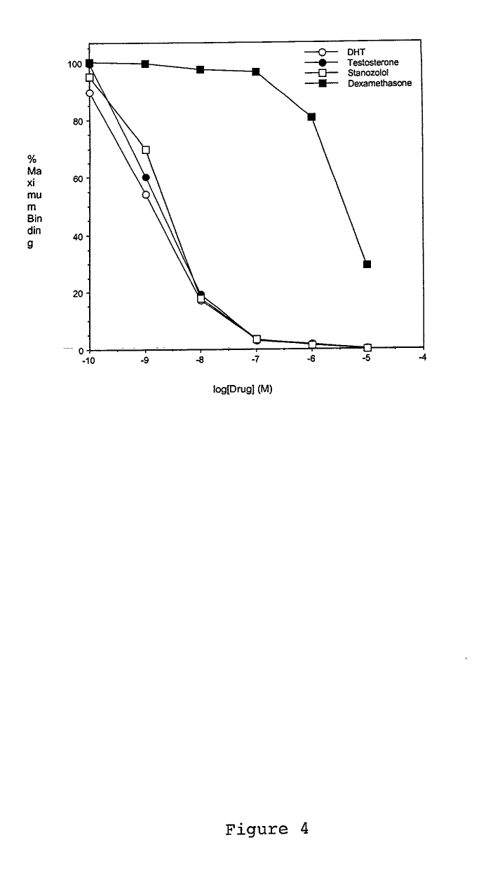

Following sequence confirmation of an open reading frame encoding an AR protein highly homologous to those reported in other species, the insert in pCR2.1-TOPO was subcloned into pcDNA3.1(+)(Invitrogen). Plasmid DNA was prepared by PerfectPrep and transiently transfected into A293S cells growing in DMEM/F12 (Life Technologies, Gaithersburg, Md.) supplemented with 10% fetal bovine serum (FBS; Gemini BioProducts, Calabasas, Calif.) in 100 mm dishes using Lipofectamine reagent (Life Technologies). 24 hr after transfection, each plate of cells was trypsinized, plated on a 500 cm² dish, and allowed to grow for 48 hours. Cell cytoplasmic extract was then prepared by sonication in binding buffer (50 mM HEPES pH 7.2, 1.5 mM EDTA, 5 mM DTT, 10 mM NaF, 10 mM Na₂MoO₄, 10% glycerol, 0.1 mg/ml bacitracin, and 1 mM Pefabloc) using 7.5 μl per cm². Binding reactions were performed at 4° C. for 1 hr in a volume of 0.2 ml containing 10 μl cell extract, 1 nM ³H-DHT ([1,2,4,5,6,7-³H]-DHT was purchased from New England Nuclear (Boston, Mass.)), plus unlabeled competitors in a dose range of 10⁻⁵ M to 10⁻¹⁰ M. The competitors used were dihydrotestosterone (DHT; 5α-androstan-17β-ol-3-one), testosterone; (4-androsten-17B-ol-3-one), and dexamthasone (9α-fluoro-16αmethylprednisolone), each purchased from Sigma Chemical Co. (St. Louis, Mo.), as well as stanozolol (17β-Hydroxy-17α-methyl-androstano[3,2-c]pyrazole, purchased from Steraloids, Inc. (Newport, R.I.)). Radioligand bound to the AR was captured on hydroxylapatite in 96-well filter plates (MultiScreen, Millipore, Bedford, Mass.). The plates were then washed three times with 4° C. wash buffer (50 mM HEPES, 10 mM PO₄, 0.1% TRITON X-100, 100 mM KCL) and the bound ³H-DHT quantitated by scintillation counting. See FIG. 4.

### Example 5

### Transactivation Assay

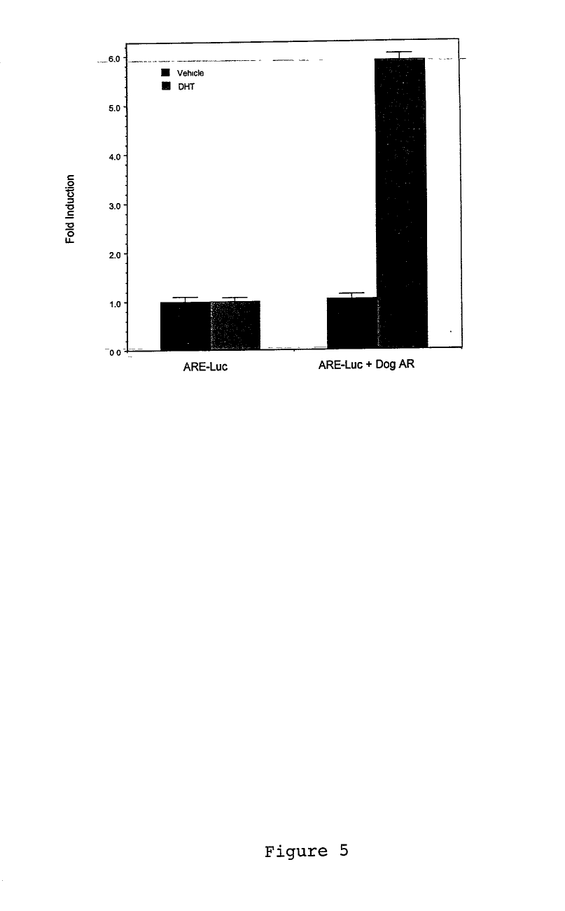

A293S cells growing in 100 mm dishes were transiently transfected with 5 μg canine AR/pcDNA3.1(+), 2 μg ARE-Luc (GGTACAAAATGTTCT-TK-Luciferase), 1 μg pEGFP-C1 (Clontech), and 1 μg CMV-β-gal per dish using Lipofectamine. Control plates were transfected with all the same plasmids except canine AR. 24 hr following transfection, each 100 mm dish was split into two 12-well dishes and the cells were treated 24 hr later with either vehicle (0.1% ethanol) or DHT (10⁻⁷ M). Cells were harvested 24 hr after compound addition and analyzed for luciferase activity (Luciferase Assay System, Promega, Madison, Wis.) and β-galactosidase activity (Galacto-Star, Tropix, Inc., Bedford, Mass.). Data is expressed as fold induction of luciferase activity as compared to vehicle, following normalization for transfection efficiency using β-galactosidase activity. This experiment demonstrated that that the cloned canine AR is capable of functionally activating an ARE in a ligand dependent manner. See FIG. 5.

### Example 6

### Northern Blot Analysis

For determination of canine AR transcript size by Northern blotting, 10 μg canine prostate poly-A⁺ RNA was separated by electrophoresis in a 0.9% agarose-1.89% formaldehyde gel, transferred to Nytran membrane (Schleicher and Schuell, Keene, N.H.) by capillary blotting in 10× SSC, and hybridized to a ³²P-labeled fragment spanning the entire canine AR protein coding region. Following hybridization and washing, AR transcripts were detected by phosphorimage analysis (Cyclone Storage Phosphor System, Packard, Meriden, Conn.). Three large transcripts (˜9 kb) of very low abundance were detected.

### Example 7

### Analysis of Canine Androgen Receptor mRNA Expression by RT-PCR

One □g total RNA isolated from canine prostate, spleen, heart, testis, liver, kidney, and skeletal muscle was reverse transcribed as described above. 2 □I of each RT reaction was then amplified by PCR using primers for canine AR (5′ primer: 5′-GACGGTGTCACACATTGAAGGC; 3′ primer: 5′-GGTTGCACAGAGTCCAGGAGC). Amplification for all tissues was performed for 24 cycles, the cycle number at which the predicted 627 bp product from canine prostate cDNA was first detectable in ethidium bromide stained gel. PCR products were resolved in a 1% agarose-TBE gel, transferred to Nytran by Southern blot under alkaline conditions, and hybridized to the ³²P-labeled canine AR probe as described herein. While not absolutely quantitative, AR mRNA was relatively highly expressed in prostate, testes, and kidney and was detectable in all other tissues examined.

### Example 8

### Immunohistochemical Localization of Canine Androgen Receptor

The ventral portion of a canine prostate was sliced into ˜2 mm sections and fixed in 4% parafomaldehyde in phosphate buffered saline(PBS). Canine proximal femur was fixed in 10% buffered formalin and decalcified in Immunocal for 12 days. Fixed tissues were embedded in paraffin, sectioned, deparaffinized with HistoClear (National Diagnostic, Atlanta, Ga.) and rehydrated through graded ethanol to saline. A polyclonal antiserum recognizing amino acids 900-918 at the C-terminus of the human AR (identical to the corresponding canine sequence) was obtained from Santa Cruz (Santa Cruz, Calif.) and used in conjunction with the HistoGold detection kit (Zymed, South San Francisco, Calif.). Following immunohistochemical detection of the anti-AR receptor antibodies, prostate sections were counterstained with a displacement connective tissue stain, bone sections were stained with Hemotoxylin and Eosin (H+E), and all sections were subsequently dehydrated through graded ethanol, mounted in Permount (Fisher, Fair Lawn, N.J.). Sections were photographed under both visible and reflected light at a final magnification of 125× or 250×. Silver grains showing the positions of AR positive cells are clearly visible under reflected light, while only a minimal background of silver grains is detected in the section where primary antibody was excluded. This experiment showed strong specific staining of the secretory epithelial cells in canine ventral prostate and specific staining of the cells of the periosteal layer, as well as osteoblasts in canine femur.

### Example 9

### ³H-DHT Competitive AR Cell Extract Preparation and Binding Analysis

Cell cytoplasmic extracts were prepared on ice. It is important that this is done as rapidly as possible. First, media was removed from cells pelleted at ˜450× g for 5 min. Supernatant was aspirated and binding buffer (50 mM HEPES pH 7.2, 10% Glycerol, 1.5 mM EDTA, 5 mM DTT, 10 mM NaF, 10 mM Na₂MoO₄, 0.1 mg/ml Bacitracin, run through a 0.2 □m filter prior to use) that contains 1 mM Pefabloc was added in a volume of 1 ml per 6.67×10⁶(=1 X) or 6.67×10⁷ cells (=10 X). This mixture was vortexed briefly and sonicated (constant, output=3) for 20-30 seconds. The resulting lysate was centrifuged at 4° C. for 10 minutes at 10,000× g. The supernatants were pooled and aliquoted into labeled tubes, frozen in liquid nitrogen and stored at −80° C. These lysates were used to peform the ³H-DHT competitive binding assay.

The cell lysates prepared above were thawed on ice. MHVB N45 plate with 45 μl column loader (Millipore #'s MHVBN4550 and MACL09645, respectively) were pre-loaded with hydroxylapatite (Calbiochem Cat# 391947). 120 □I of the cell lysate/binding buffer mix was aliquoted into all wells of the MHVB N45 plate at a predetermined extract dilution. 80 □I of 2.5X “Hot” mix (2.5 nM ³H-DHT in binding buffer), with or without competitor, was added to each well, followed by incubation for 1 hour at 4° C. Test and control compounds were added at 10⁻⁶ to 10⁻¹¹ M and tested in triplicate. The plate was transferred to a vacuum manifold (Millipore # MAVM09601) and gentle vacuum applied. The vacuum was removed once wells were dry, 250 □I Wash Buffer (or WB, 50 mM HEPES pH 7.2, 10 mM sodium phosphate buffer pH 7.2, 0.1% Triton X-100, and 100 mM KCl) at 4° C. was added to all wells and vacuum was re-applied. Washing with WB was repeated a total of three times. After last wash, vacuum was applied to plate until it was completely dry. The plate bottom was removed, and the top of the plate placed in an adapter (Packard #6005178), and 50 □I MicroScint 20 (Packard #6013621) added to each well. Plates were covered with TopSeal-A (Packard, #6005185), stored in dark 1 hr, and counted in Packard TopCount (protocol #34).

All publications and patents mentioned in the above specification are herein incorporated by reference. Various modifications and variations of the described invention will be apparent to those skilled in the art without departing from the scope and spirit of the invention. Although the invention has been described in connection with specific preferred embodiments, it should be understood that the invention as claimed is not limited to such specific embodiments.

That which is claimed is:

1. An isolated proteinaceous molecule having canine AR activity, wherein said proteinaceous molecule comprises an amino acid sequence of SEQ ID NO:2, or an amino acid sequence of SEQ ID NO:2 with one or more conservative substitutions therein.

2. An isolated proteinaceous molecule according to claim 1, wherein said amino acid sequence has zero to three conservative substitutions therein.

3. An isolated proteinaceous molecule according to claim 1, wherein said amino acid sequence comprises an amino acid sequence of SEQ ID NO:2.

4. An isolated DNA molecule encoding a proteinaceous molecule according to claim 1.

5. An isolated DNA molecule encoding a proteinaceous molecule according to claim 3.

6. A recombinant expression vector comprising a DNA molecule according to claim 4.

7. A cell transformed by an expression vector according to claim 6.

8. A cell according to claim 7, wherein said cell is that of a prokaryote.

9. A cell according to claim 7, wherein said cell is that of a eukaryote.

10. A cell according to claim 9, wherein said eukaryote is yeast.

11. A cell according to claim 9, wherein said eukaryote is a mammal.

12. A cell according to claim 11, which is a CHO cell.

13. A method of producing a proteinaceous molecule having an activity of canine AR, which comprises culturing a cell according to claim 7 for a time sufficient to produce said proteinaceous molecule.

14. A pharmaceutical composition comprising the proteinaceous molecule according to claim 1 and a pharmaceutically acceptable carrier therefor.

15. A pharmaceutical composition comprising the proteinaceous molecule according to claim 3 and a pharmaceutically acceptable carrier therefor.

16. A specific binding partner that selectively binds to the proteinaceous molecule according to claim 1.

17. A method of discovering ligands for the canine AR protein, said method comprising the steps of:
a) combining a test substance with the proteinaceous molecule according to claim 1;
b) measuring specific binding between said test substance and said proteinaceous molecule; and
c) classifying as a ligand said test substance if it binds to said proteinaceous molecule.

18. A method of discovering modulators of canine AR protein activity, said method comprising the steps of:
a) combining a test substance with the proteinaceous molecule according to claim 1;
b) measuring the activity of said proteinaceous molecule in the presence and absence of said test substance and; and
c) classifying said test substance as a modulator of canine AR activity if it modulates the activity of said proteinaceous molecule.
